(19) United States
(12) Patent Application Publication
(10) Pub. No.: US 2026/0174895 A1
(43) Pub. Date: Jun. 25, 2026

(54) METHOD FOR DELIVERY OF AN AGENT ACROSS BIOLOGICAL BARRIERS

(71) Applicant: TZU CHI UNIVERSITY, Hualien City (TW)

(72) Inventors: Hsin-Hou CHANG, Hualien City (TW); Der-Shan SUN, Hualien City (TW)

(21) Appl. No.: 18/989,469

(22) Filed: Dec. 20, 2024

(51) Int. Cl. A61K 47/69; A61K 47/64; A61P 25/22; A61P 25/24; C12N 15/113

(52) U.S. Cl. CPC A61K 47/6909; A61K 47/64; A61P 25/22; A61P 25/24; C12N 15/1137; C12N 2310/14; C12N 2320/32

(57) ABSTRACT

The present disclosure provides a protein-vesicle conjugate, comprising one or more secretory proteins or a fragment thereof expressed on or conjugated to a surface of a vesicle and optionally an agent, and a method for delivery of an agent with the protein-vesicle conjugate across a biological barrier to a cell and tissue containing the cell.

### FIELD OF THE INVENTION

The present invention is related to the field of drug delivery to cells, and particularly to delivery of drugs across a biological barrier to a cell with protein-vesicle conjugate.

### BACKGROUND OF THE INVENTION

The blood-brain barrier (BBB) constitutes a specialized vascular system in the brain maintaining central nervous system (CNS) homeostasis by tightly regulating passage of specific nutrients therethrough and preventing entry to harmful substances like neurotoxic agents. However, the presence of the BBB poses challenges for treatment of CNS diseases, restricting access by certain drugs and large-molecule therapeutics, including biopharmaceuticals, into the brain. For instance, after administration, concentration of therapeutic antibodies in the brain is typically only 0.01-0.1% of that in plasma. Although even these low levels of antibodies can produce pharmacological effects, there is a clear advantage to achieving higher concentrations thereof, which would reduce the required dosage and improve therapeutic window.

### SUMMARY OF THE INVENTION

In one aspect, the present disclosure provides a method for delivery of an agent across a biological barrier to a cell and/or a tissue containing the cell, comprising contacting a protein-vesicle conjugate or a pharmaceutical composition comprising the protein-vesicle conjugate with a cell and/or a tissue of a subject, wherein the protein-vesicle conjugate comprises: (a) one or more secretory proteins or a fragment thereof expressed on or conjugated to a surface of a vesicle, and/or an antibodies specific to the one or more secretory proteins or the fragment thereof and expressed on or conjugated to the surface of the vesicle; and (b) an agent embedded in the vesicle. In one embodiment, the the present disclosure provides a method for delivery of an agent across a biological barrier to a cell and/or a tissue containing the cell, comprising contacting a protein-vesicle conjugate or a pharmaceutical composition comprising the protein-vesicle conjugate with a cell and/or a tissue of a subject, wherein the protein-vesicle conjugate comprises: (a) one or more secretory proteins or a fragment thereof expressed on or conjugated to a surface of a vesicle and (b) an agent embedded in the vesicle. In another embodiment, the the present disclosure provides a method for delivery of an agent across a biological barrier to a cell and/or a tissue containing the cell, comprising contacting a protein-vesicle conjugate or a pharmaceutical composition comprising the protein-vesicle conjugate with a cell and/or a tissue of a subject, wherein the protein-vesicle conjugate comprises: (a) an antibodies specific to the one or more secretory proteins or the fragment thereof and expressed on or conjugated to the surface of the vesicle; and (b) an agent embedded in the vesicle.

In some embodiments, the biological barrier described herein includes, but is not limited to, intestinal barrier, nasal barrier, pulmonary barrier, blood-brain barrier (BBB), and skin barrier. In some embodiments, the cell or the tissue containing the cell can be in the central nervous system of the subject. For example, the cell or the tissue containing the cell can be in brain of the subject. The biological barrier can be BBB.

In some embodiments, the cell described herein can be an inflamed and/or injured cell, which undergoes regulated cell death (apoptotic and non-apoptotic cell death). In some embodiments, the cell or the tissue containing the cell is in central nervous system of the subject. In some embodiments, the cell or the tissue containing the cell is in brain of the subject. In some embodiments, the cell may be a neuron, a microglia cell, or an astrocyte.

In some embodiments, before administration of the protein-vesicle conjugate, the method further comprises a step of inducing and/or administering the inflamed and/or injured cell within the BBB. While not wishing to be bound by any particular theory, it is believed that since an inflammation and/or injury occurred in the brain, the BBB integrity may be disrupted, thus the protein-vesicle conjugate can penetrate therethrough more easily.

The protein-vesicle conjugate described herein comprises one or more secretory proteins or a fragment thereof expressed on or conjugated to the surface of the vesicle and optionally an agent.

Examples of the one or more secretory proteins or the fragment thereof described herein include, but are not limited to, P-selectin (P-sel), cation-independent mannose 6-phosphate receptor (CI-MPR), L-selectin (L-sel), E-selectin (E-sel), P-selectin glycoprotein ligand-1 (PSGL-1), CD22 (siglec2), galectin-3 (Gal-3), Klotho, pentraxin 3 (PTX3), CD47, CD42b, dendritic cell-specific intercellular adhesion molecule-3-grabbing non-integrin (DC-SIGN) receptor (DC-SIGNR), intercellular adhesion molecule 1 (ICAM-1; CD54), vascular cell adhesion molecule (VCAM), integrin α4β1, VE-caderin, annexin V, toll-like receptor 3 (TLR3), programmed cell death 1 ligand 1 (PD-L1), IL-1β, IL-1RA, TNF-α, etanercept (soluble TNF receptor), EGF, fibroblast growth factor (FGF) 23, insulin, glial cell-line derived neurotrophic factor (GDNF), BDNF, β-NGF, NT-3, TGF-β1, activin A, BMP4, BMP6, BMP9, BMP-10, GDF-8, GDF-10, GDF-11, and a fragment thereof. Examples of the one or more antibodies specific to the one or more secretory proteins or the fragment thereof described herein include, but are not limited to, anti-PSGL-1 immunoglobulin (anti-PSGL-1 Ig) and anti-PDL1 Ig.

In some embodiments, the one or more secretory proteins or the fragment thereof described herein is selected from P-selectin, b-NGF, NT3, TGF-b1, activin A, BMP4, BMP6, BMP9, BMP10, and a fragment thereof.

In some embodiments, the cell described herein is a neuron, and the one or more secretory proteins or the fragment thereof is selected from P-selectin, activin A, BMP9, and a fragment thereof.

In some embodiments, the cell described herein is a microglia cell, and the one or more secretory proteins or the fragment thereof is selected from P-selectin, β-NGF, NT3, activin A, and a fragment thereof.

In some embodiments, the cell described herein is an astrocyte, and the one or more secretory proteins or the fragment thereof is selected from P-selectin, β-NGF, NT3, activin A, BMP6, BMP9, and a fragment thereof.

In some embodiments, the protein-vesicle conjugate described herein comprises CI-MPR in combination with Klotho, CI-MPR in combination with P-selectin, or Klotho in combination with P-selectin.

In some embodiments, the vesicle described herein is a liposome or a micelle.

In some embodiments, the agent described herein is separated from the secretory protein s or the fragment thereof and encapsulated within the vesicle or attach to an outer surface of the vesicle.

In some embodiments, the agent described herein is a diagnostic contrast agent, a cell survival enhancing agent, a cell survival suppressing agent, a cell component, an organelle, a cell, a cytotoxic agent, an antitumor drug, a toxin or an antibody, a lipid, a protein, DNA, RNA, a therapeutic agent or a nanomaterial.

### BRIEF DESCRIPTION OF DRAWINGS

FIGS. 1A to 1C presents a quantitative analysis of relative fluorescence levels in a mouse brain. Fluorescein-loaded liposomes conjugated with different secretory proteins on their surfaces were tested for their ability to cross the BBB and stain the mouse brain after a 20-hour restraint stress protocol (FIGS. 1A, and 1B, brain fluorescence levels; FIG. 1C, experiment outline and settings). Vehicle injection, along with injections of BSA-and IgG-Fc-conjugated liposomes, was used as negative controls. The fluorescent levels in the mouse brain of BSA groups were normalized to 1-fold (horizontal line). We used single protein conjugation alone and employed P-selectin (P-sel) and the cation-independent mannose-6-phosphate receptor (CI-MPR) as positive controls. Relevant brain images are available in FIGS. 2A-2F.

FIGS. 2A to 2F are representative images of fluorescent dye levels delivered into the mouse brain via the delivery system. The upper panels show the secretory proteins conjugated with the liposomes, while the lower panels present the corresponding brain images. The corresponding quantitative analysis results are shown in FIGS. 1A to 1C.

FIGS. 3A to 3D illustrate the targeted rescue of inflamed and injured brain cells using secretory protein-conjugated liposomes loaded with caspase-1 siRNA. FIG. 3A provides an overview of the experiment. The markers used to label neurons, microglia, astrocytes, and pyroptosis signal in the flow cytometry analyses were NeuN, F4/80, GFAP, and the activated form of caspase-1, respectively (FIG. 3A). FIGS. 3B, 3C, and 3D show the relative pyroptosis levels (a form of regulated cell death) in neurons, microglia, and astrocytes, respectively, in the mouse brain under restraint stress conditions compared to controls. #P<0.05 vs. BSA groups, indicating significant rescue. These findings align with the fluorescein delivery experiments (FIG. 1), where P-selectin, b-NGF, NT3, TGF-b1, activin A, BMP4, BMP6, BMP9, and BMP10 demonstrate effective drug-delivery effect in neurons (B), microglia cells (C), and astrocytes (D) in the mouse brain following restraint stress. Additionally, siRNA is an ideal cargo for this drug-delivery system, as demonstrated in this experiment with caspase-1 siRNA, which effectively ameliorates pyroptosis and brain inflammation induced by restraint stress in mice.

FIG. 4 shows direction of liposomes conjugated with secretory proteins toward inflamed and damaged brain cells. Selected secretory proteins include cation-independent mannose-6-phosphate receptor (CI-MPR), Klotho, and P-selectin (P-sel), while bovine serum albumin (BSA) served as a non-lectin negative control protein. Vehicle indicated treatments using solvents without fluorescent liposomes. The upper panel shows fluorescent images of the mouse brain following restraint stress, and the lower panel illustrates treatments and corresponding relative fluorescence levels (numbers). The conjugation of liposomes with a combination of effective secretory proteins can produce a synergistic effect in fluorescence dye delivery (lower panel; e.g., combined use of CI-MPR, Klotho, and P-sel).”

FIGS. 5A to 5C show targeting of inflamed and injured cells in the brain using caspase-3 inhibitor loaded secretory protein-conjugated liposomes. FIG. 5A is an experimental overview. FIGS. 5B and 5C show assessment of relative IL-1 levels in the mouse brain under restraint stress conditions compared to controls. Notably, only intravenous administration of caspase-3 inhibitor (z-DEVD-FMK) loaded P-selectin-conjugated liposomes [P-sel-lipo(C3in)], but not bovine serum albumin (BSA)-conjugated liposomes, significantly mitigated the elevated levels of the pro-inflammatory cytokine IL-1β induced by restraint stress (*P<0.05, compared to respective untreated groups; #P<0.05, P-sel-lipo (C3in) vs. vehicle groups) [P-sel-lipo(C3in): P-selectin-conjugated liposomes loaded with caspase 3 inhibitor Z-DEVD-FMK].

FIGS. 6A to 6C show that the administration of caspase-1 inhibitor-loaded P-selectin-conjugated liposomes effectively improved LPS-induced anxiety- and depression-prone behavior. Image records of mouse tracks in the open field test are presented in FIG. 6A. Quantitative analyses of mouse behavior are displayed in FIGS. 6B and 6C, revealing a significant alleviation of anxiety-and depression-prone behavior induced by LPS following intravenous treatment with P-selectin-conjugated liposomes loaded with caspase-1 inhibitor z-WEHD-FMK [P-sel-lipo (C1in)]. Statistical significance was denoted as * for P<0.05 compared to respective untreated groups (UN), and # for P<0.05 comparing P-selectin-conjugated liposomes loaded with caspase-1 inhibitor (P-sel-lipo (C1in)) with vehicle groups. [P-sel-lipo(C1in): P-selectin-conjugated liposomes loaded with caspase-1 inhibitor z-WEHD-FMK].

FIGS. 7A to 7K show significant improvement in restraint stress-induced anxiety- and depression-like behavior upon treatment with caspase-1 inhibitor-loaded liposomes conjugated with P-selectin. The results of the open arm test (FIGS. 7A-7F) and tail suspension test (FIGS. 7G-7K) are presented. Representative image records of mouse tracks in the open arm test (FIGS. 7A-7D) and tail suspension test (FIGS. 7G-7J) are displayed. Quantitative analyses of mouse behavior (FIGS. 7E and 7F) demonstrated a marked amelioration of restraint stress-induced anxiety and depression-like behavior following intravenous treatment with P-selectin-conjugated liposomes loaded with caspase-1 inhibitor z-WEHD-FMK [P-sel-lipo (C1in)]. Statistical significance was denoted as * for P<0.05 compared to respective untreated groups (UN), and # for P<0.05 comparing P-selectin-conjugated liposomes loaded with caspase-1 inhibitor [P-sel-lipo (C1in)] with vehicle groups. [P-sel-lipo(C1in): P-selectin-conjugated liposomes loaded with caspase-1 inhibitor z-WEHD-FMK].

FIG. 8 shows microscopic images of mouse brain sections captured using fluorescence microscopy. In the absence of Cre-recombinase expression, brain cells of transgenic mice [B6.129-(Gt(ROSA)26Sor^{tm4(ACTB-tdTomato, EGFP)Luo}/J] express only the red fluorescence protein, tdTomato, without expressing enhanced green fluorescence protein (EGFP). However, when functional Cre-recombinase is introduced into these brain cells, it catalyzes the LoxP sites in the transgene, switching off tdTomato red fluorescence expression and activating EGFP expression, resulting in green fluorescence replacing the red. Our findings demonstrated the expression of EGFP in brain cells (represented by the green spots) of transgenic mice [B6.129-(Gt(ROSA)26Sor tm4(ACTB tdTomato, EGFP)Luo/] that underwent restraint stress. This EGFP expression was observed following the delivery of functional recombinant protein of Cre-recombinase using secretory protein P-selectin-conjugated liposomes. These results indicate that this delivery system is well-suited for the efficient delivery of functional enzymes and proteins, such as those used in enzyme replacement therapy, to brain cells without apparent lysosome digestion.

FIGS. 9A to 9H show significant improvement in LPS-induced anxiety- and depression-like behavior upon treatment with P-selectin-conjugated liposomes loaded with caspase-1 siRNA. The experimental outline is presented in FIG. 9A. Image records of mouse tracks in the open field test are shown in FIGS. 9B-9E. Quantitative analyses of mouse behavior are displayed in FIGS. 9F-9H, revealing a notable amelioration of anxiety-and depression-like behavior induced by restraint stress following intravenous treatment with P-selectin-conjugated liposomes loaded with caspase-1 siRNA, in contrast to the control groups treated with BSA-conjugated liposomes loaded with caspase-1 siRNA. Statistical significance was denoted as * for P<0.05 compared to respective untreated groups (UN), and # for P<0.05 comparing P-selectin-conjugated liposomes loaded with caspase-1 siRNA (P-sel-lipo) with vehicle groups. (P-sel-lipo: P-selectin-conjugated liposomes loaded with caspase-1 siRNA; BSA-lipo: BSA-conjugated liposomes loaded with caspase-1 siRNA).

### DETAILED DESCRIPTION OF THE INVENTION

Where the definition of terms departs from common use, applicant intends to utilize the following definitions, unless specifically indicated.

As used in this specification and the appended claims, the singular forms “a,” “an” and “the” include plural referents unless the content clearly dictates otherwise.

As used herein, the use of “or” means “and/or” unless stated otherwise. In the context of a multiple dependent claim, the use of “or” refers back to more than one preceding independent or dependent claim in the alternative only.

As used herein, the term “one or more” is readily understood by one of ordinary skill, particularly when read in context of its usage.

As used herein, the term “secretory protein” may refer to any protein secreted by a cell. Secretory proteins may include hormones, enzymes, toxins, and antimicrobial peptides. In some embodiments, the secretory proteins are lectins.

As used herein, the term “liposome” is a generic term encompassing a variety of single and multilamellar lipid vehicles formed by the generation of enclosed lipid bilayers or aggregates. Liposomes may be characterized as having vesicular structures with a bilayer membrane, generally comprising a phospholipid, and an inner medium that generally comprises an aqueous composition.

As used herein the term “micelle” refers to an aggregate (or supramolecular assembly) of surfactant molecules dispersed in a liquid colloid. A typical micelle in aqueous solution forms an aggregate with the hydrophilic “head” regions in contact with surrounding solvent, sequestering the hydrophobic single-tail regions in the micelle center.

As used herein, the terms “agent” or “therapeutic agent” refers to an agent capable of treating and/or ameliorating a condition or disease.

As interchangeably used herein, the terms “individual,” “subject,” “host,” and “patient,” refer to a mammal, including, but not limited to, murines (rats, mice), non-human primates, humans, canines, felines, ungulates (e.g., equines, bovines, ovines, porcines, caprines), etc.

As used herein, the term “therapeutically effective amount” or “efficacious amount” refers to the amount of the vesicle that, when administered to a mammal or other subject for treating a disease, is sufficient to affect such treatment for the disease.

As used herein, the terms “treatment,” “treating,” and the like, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

As used herein, the term “conjugation site” refers to the site where a covalent linkage is formed between two macromolecules, mostly terminal-to-sidechain branched conjugations, and occasionally molecular head-to-tail linear conjugations.

The present disclosure demonstrates that drug delivery across a biological barrier can be accomplished using the secretory protein-conjugated vesicles recited herein. Through the use of these vesicles, it successfully delivers various molecules, including small molecule drugs, fluorescent compounds, nanoparticles, proteins, nucleic acids, and combinations thereof, to cells. This highlights the potential of secretory protein-conjugated vesicles as an effective means of delivering a diverse range of substances to the cells.

Apoptosis is controlled by the integration of multiple pro-and anti-apoptotic signals. The delivery of an agent or a therapeutic agent with the vesicle of the invention to apoptotic cells is directed to a disease associated with apoptosis alteration. The disease associated with apoptosis alteration can include but is not limited to trauma, exposure to chemical and physical toxic factors, genetic disease, age-related disease, age-related disease, cardiovascular disease, infectious disease, neoplastic disease, neurodegenerative disease, metabolic disease, aging, obesity, cancer, neurodegeneration induced by β-amyloid or α-synuclein (Alzheimer, Parkinson, Huntington, amyotrophic lateral sclerosis) or toxicity, myodegenerative conditions, or chronic lung inflammation caused by cystic fibrosis, cardiovascular disorder (such as ischemia, heart failure and infectious disease) and autoimmune disease (systemic lupus erythematosus, autoimmune lymphoproliferative syndrome, rheumatoid arthritis and thyroiditis).

Autophagy is a lysosomal degradation pathway essential for survival, differentiation, development, and homeostasis. The delivery of an agent or therapeutic agent with the vesicle of the invention to autophagic cells is directed to a disease associated with autophagy deregulation. The disease associated with autophagy deregulation includes but is not limited to trauma, exposure to chemical and physical toxic factors, genetic disease, age-related disease, cardiovascular disease, infectious disease, neoplastic disease, neurodegenerative disease, metabolic disease, aging (when ATG5 is overexpressed in the entire organism), obesity (when ATG7 or the pro-autophagic transcription factor EB [TFEB] are overexpressed in hepatocytes), cancer (when beclin 1 is expressed in KRAS-induced lung adenomas), neurodegeneration induced by β-amyloid or α-synuclein or toxicity (when TFEB or beclin 1 are overexpressed in the brain or when cystatin B, an inhibitor of lysosomal cysteine proteases, is knocked out), myodegenerative conditions (when TFEB or beclin 1 are targeted to the skeletal muscle), and chronic lung inflammation caused by cystic fibrosis (when beclin 1 is expressed in the lung).

Pyroptosis is a form of regulated cell death characterized by the activation of inflammatory caspases, leading to cell lysis and the release of pro-inflammatory cytokines like IL-1β and IL-18. This process is mediated by the formation of gasdermin pores in the cell membrane. In the central nervous system (CNS), pyroptosis has been implicated in various neurodegenerative and neuroinflammatory diseases. For example, in Alzheimer's disease, pyroptosis contributes to neuronal death and the release of inflammatory mediators, exacerbating amyloid-beta plaque formation and tau pathology. In multiple sclerosis, pyroptosis in microglia and astrocytes promotes demyelination and neuronal damage. Additionally, in traumatic brain injury, pyroptosis-induced inflammation aggravates neuronal damage and impairs recovery. These examples highlight the critical role of pyroptosis in CNS diseases, making it a potential target for therapeutic interventions.

The present disclosure provides a method for delivery of an agent across a biological barrier to a cell and/or a tissue containing the cell, comprising contacting a protein-vesicle conjugate or a pharmaceutical comprising the protein-vesicle conjugate with a cell and/or a tissue of a subject, wherein the protein-vesicle conjugate comprises: (a) one or more secretory proteins or a fragment thereof expressed on or conjugated to a surface of a vesicle, and/or one or more antibodies specific to the one or more secretory proteins or the fragment thereof and expressed on or conjugated to the surface of the vesicle; and (b) an agent embedded in the vesicle.

Liposomes provided herein include unilamellar liposomes, multilamellar liposomes and multivesicular liposomes. Liposomes provided herein may be composed of positively charged, negatively charged or neutral phospholipids.

A liposome used in the invention can be provided by various known methods. For example, a phospholipid such as the neutral phospholipid dioleoylphosphatidylcholine (DOPC), Dipalmitoyl Phosphatidylcholine (DPPC) and/or EPC, can be dissolved in alcohol or other organic solvent and then mixed with a component for inclusion in the lipid bilayer. The mixture may further include various detergents. Typically, a lipid mixture is vortexed, frozen in a dry ice/acetone bath, and lyophilized overnight. The lyophilized preparation is stored at −20° C. or less for extended periods of time. When required the lyophilized liposomes are reconstituted.

Alternatively, a liposome can be prepared by mixing lipids in a solvent in a container, e.g., a glass, pear-shaped flask. The container should have a volume ten-times greater than the volume of the expected suspension of liposomes. Using a rotary evaporator, the solvent is removed at approximately 40° C. under negative pressure. The solvent normally is removed within about 5 minutes to 2 hours, depending on the desired volume of the liposomes. The composition can be dried further in a desiccator under vacuum. The dried lipids generally are discarded after about 1 week because of a tendency to deteriorate with time.

Micelle structure will itself be determined, in large part, by the types and compositions of polymer molecules used to form the micelle and the solvent environment of the micelle. In some embodiments, micelles are fabricated using non-ionic triblock co-polymers consisting of both hydrophilic and hydrophobic monomer units. In embodiments of the present disclosure, a poloxamer, a triblock copolymer of poly (ethylene oxide)-poly(propylene oxide)-poly(ethylene oxide) (PEO-PPO-PEO) is used. In some embodiments, the micelles of this disclosure can be prepared using PEG-PLA polymers of a variety of block sizes (e.g., a block size within a range as noted) and in a variety of ratios (e.g., PEG: PLA of about 1:10 to about 10:1, or any integer ratio within said range).

In some embodiments, the protein-vesicle conjugate can be artificially engineered or cell-derived.

The secretory protein described herein may be conjugated into the vesicle through a supplement of functional-group labeled lipid using shear force-based methods (Yu B, Lee R J, Lee L J. Microfluidic methods for production of liposomes. Methods Enzymol. 2009; 465:129-141; Jeong D, Jo W, Yoon J, et al. Nanovesicles engineered from ES cells for enhanced cell proliferation. Biomaterials. 2014; 35(34):9302-9310).

In some embodiments, the therapeutic agent is a cell survival enhancing agent (or a cell death suppressing agent), which is able to conduct a drug-mediated rescue of tissue injury. In some embodiments, the agent is a cell survival suppressing agent, cell death enhancing agent or antitumor agent, which is able to reduce target cell survival of those tissues containing naturally occurred autophagy and apoptotic cells such as tumors or reduce the selected specific tissue wherein the autophagy and apoptotic cells are artificially induced in the specific tissues using cytotoxic agents such as a drug, a toxin or an antibody against tissue-specific proteins.

In one embodiment, the agent is a diagnostic agent or a therapeutic agent. In one embodiment, the agent is an autophagic or apoptotic drug. In some embodiments, examples of the agent include, but are not limited to, an anti-pathogen drug, an autophagy inhibitor, an enzyme inhibitor, a cell signaling inhibitor, a diagnostic contrast agent, a cell survival enhancing agent (or a cell death suppressing agent), a cell survival suppressing agent (or a cell death enhancing agent), a cell (such as stem cell and progenitor cell), a cell component, an organelle, a cytotoxic agent, an antitumor drug, a toxin or an antibody a lipid, a protein, DNA, RNA, a therapeutic agent and a nanomaterial. In one embodiment, the antagonist of the aforementioned first protein or second protein (such as the soluble form, corresponding ligand and the neutralizing and blocking antibody) is able to serve as antidotes to reduce the vesicle-targeting to autophagic and apoptotic cells and autophagic and apoptotic cells-containing tissues. In some embodiments, the agent is bardoxolone methyl, chloroquine, quinine, hydrochloroquine, sorafenib, sunitinib, Hsp90 inhibitor, metformin or crizotinib. In some embodiments, the RNA may be one or more of small interfering RNA (siRNA), small hairpin RNAs (shRNA), microRNA (miRNA), messenger RNA (mRNA), transfer RNA (tRNA), and ribosomal RNA (rRNA), antisense RNA, guide RNA (gRNA), ribozyme and RNA aptamers.

In some embodiments, the agent is a stem cell or a progenitor cell. The delivery of stem cells and progenitor cells can exert protective physiological function and rescue autophagic and apoptotic cell-containing tissues.

In some embodiments, the protein-vesicle conjugate or pharmaceutical composition of the invention can be administered to the patient in various ways, including topically, parenterally, intravenously, intradermally, subcutaneously, intramuscularly, colonically, rectally or intraperitoneally. Preferably, the pharmaceutical compositions are administered parenterally, topically, intravenously, intramuscularly, subcutaneously, orally, or nasally, such as via inhalation.

The protein-vesicle conjugate may be formulated as a pharmaceutical composition, and may be formulated with one or more pharmaceutically acceptable carriers. The protein-vesicle conjugate can be formulated in a variety of different manners known to one of ordinary skill. Pharmaceutically acceptable carriers are determined in part by the particular composition being administered, as well as by the particular method used to administer the composition. Accordingly, a wide variety of suitable formulations of pharmaceutical compositions of the present invention (see, e.g., Remington's Pharmaceutical Sciences, 20.sup.th ed., 2003, supra) exist. Effective formulations include oral and nasal formulations, formulations for parenteral administration, and compositions formulated for extended release.

For purposes of administration, for example, parenteral administration, sterile aqueous solutions of water-soluble salts (e.g., NaCl) can be employed. Additional or alternative carriers may include sesame or peanut oil, as well as aqueous propylene glycol. Aqueous solutions may be suitably buffered, if necessary, and the liquid diluent can first be rendered isotonic with sufficient saline or glucose. These aqueous solutions are especially suitable for intravenous, intramuscular, subcutaneous, intraperitoneal, and intratumoral (IT) injection.

Formulations suitable for oral administration can consist of liquid solutions, such as an effective amount of a compound of the present invention suspended in diluents, such as water, saline or PEG 400, capsules, sachets, depots or tablets, each containing a predetermined amount of the active ingredient, as liquids, solids, granules or gelatin, suspensions in an appropriate liquid, suitable emulsions, and patches. The liquid solutions described can be sterile solutions. The pharmaceutical forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

In some embodiments, the protein-vesicle conjugate may be administrated with one or more antagonists to the one or more secretory proteins or a fragment thereof. In some embodiments, the antagonists to the one or more secretory proteins or a fragment thereof are soluble forms of the secretory proteins or fragments thereof, corresponding ligands to the secretory proteins or fragments thereof, or antibodies to the secretory proteins or fragments thereof.

In some embodiments, the method is directed to a disease associated with regulated cell death of brain cell alteration, such as trauma, exposure to chemical and physical toxic factors, genetic disease, age-related disease, cardiovascular disease, infectious disease, neoplastic disease, neurodegenerative disease, metabolic disease, aging, obesity, cancer, inflammation, psychological stress, stroke, neurodegeneration induced by β-amyloid or α-synuclein or toxicity, myodegenerative conditions, or chronic lung inflammation caused by cystic fibrosis, or autoimmune disease. The neurodegenerative disorder may be brain fog, Alzheimer's disease, Parkinson's disease, Huntington's disease, Amyotrophic Lateral Sclerosis or stroke, the cardiovascular disorder associated brain damage is ischemia, heart failure or infectious disease and the autoimmune disease is systemic lupus erythematosus, autoimmune lymphoproliferative syndrome, rheumatoid arthritis or thyroiditis.

Although the invention has been described with reference to preferred embodiments and examples thereof, the scope of the present invention is not limited only to those described embodiments. As will be apparent to persons skilled in the art, modifications and adaptations to the described invention can be made without departing from the spirit and scope of the invention, which is defined and circumscribed by the appended claims. The following examples are provided for the intent of illustrating embodiments and advantages of the invention and are not intended to limit the scope thereof.

### Example 1: Preparation of Liposomes Engineered with Protein Conjugates

The preparation of liposomes. The liposomes were prepared by liposome kits (Sigma-Aldrich Co.) and respective lipids. Surface proteins were conjugated to the liposomes by incorporating functional-group labeled lipids and utilizing shear force-based methods (Yu, B.; Lee, R. J.; Lee, L. J. Microfluidic methods for production of liposomes. Methods Enzymol 2009, 465, 129-141, doi:10.1016/S0076-6879(09)65007-2; Jeong, D.; Jo, W.; Yoon, J.; Kim, J.; Gianchandani, S.; Gho, Y. S.; Park, J. Nanovesicles engineered from ES cells for enhanced cell proliferation. Biomaterials 2014, 35, 9302-9310, doi:10.1016/j.biomaterials.2014.07.047). The protein conjugation procedure followed the manufacturer's provided methods.

Synthesis and conjugation of liposomes. The liposomes were synthesized using established methods as described previously (Chen, Y. L.; Chen, Y. S.; Chan, H.; Tseng, Y. H.; Yang, S. R.; Tsai, H. Y.; Liu, H. Y.; Sun, D. S.; Chang, H. H. The use of nanoscale visible light-responsive photocatalyst TiO2-Pt for the elimination of soil-borne pathogens. PloS one 2012, 7, e31212, doi:10.1371/journal.pone.0031212; Wu, C. H.; Kuo, Y. H.; Hong, R. L.; Wu, H. C. alpha-Enolase-binding peptide enhances drug delivery efficiency and therapeutic efficacy against colorectal cancer. Sci Transl Med 2015, 7, 290ra291, doi:10.1126/scitranslmed.aaa 9391).

The recombinant proteins. The recombinant proteins used in this study, such as recombinant MPRs (cation-independent mannose-6-phosphate receptor: CI-MPR; cation-dependent mannose-6-phosphate receptor: CD-MPR), were obtained from R and D Systems Co.

### Example 2: Liposomes Engineered with Protein Conjugates can Successfully Deliver the Fluorescent Dye Into the Inflamed Mouse Brain After Restraint Stress

Restraint stress mouse model. A restraint stress mouse model was employed to induce psychological stress, brain inflammation, and depression and anxiety behaviors. The restraint stress procedure followed previously described methods (Chu, X.; Zhou, Y.; Hu, Z.; Lou, J.; Song, W.; Li, J.; Liang, X.; Chen, C.; Wang, S.; Yang, B., et al. 24-hour-restraint stress induces long-term depressive-like phenotypes in mice. Sci Rep 2016, 6, 32935, doi:10.1038/srep32935; Pethaperumal, S.; Hung, S. C.; Lien, T. S.; Sun, D. S.; Chang, H. H. P-Selectin is a Critical Factor for Platelet-Mediated Protection on Restraint Stress-Induced Gastrointestinal Injury in Mice. Int J Mol Sci 2022, 23, doi:10.3390/ijms231911909; Chuang, D. J.; Pethaperumal, S.; Siwakoti, B.; Chien, H. J.; Cheng, C. F.; Hung, S. C.; Lien, T. S.; Sun, D. S.; Chang, H. H. Activating Transcription Factor 3 Protects against Restraint Stress-Induced Gastrointestinal Injury in Mice. Cells 2021, 10, doi:10.3390/cells10123530). During the stress period, both the stressed and unstressed groups of mice were deprived of access to food and water. For the behavior test, the restraint stress was conducted for a duration of 20 hours. After the termination of restraint stress, both the stressed and unstressed groups of mice were given a 2-hour period to access food and water to restore their resources.

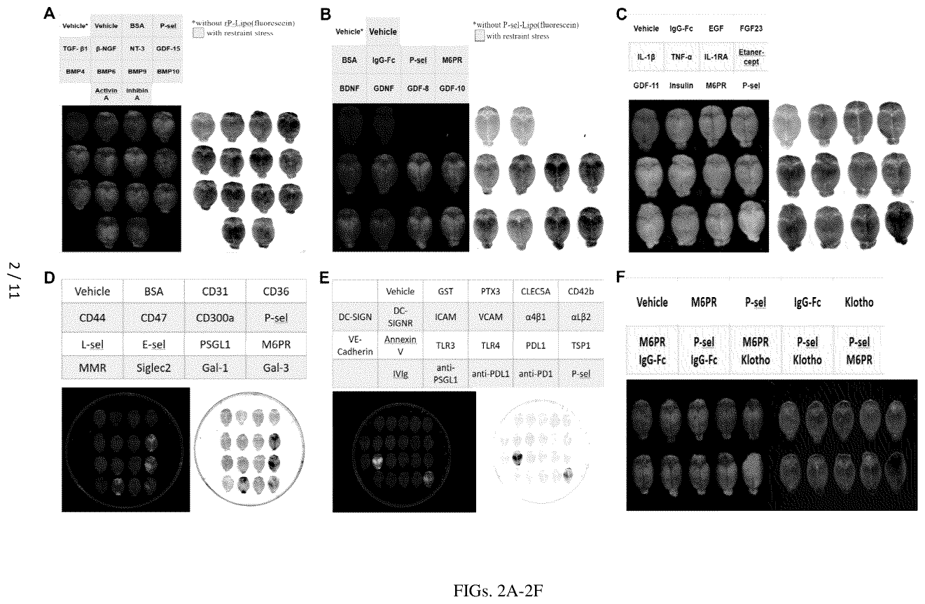

Specialized liposomes, engineered with protein conjugates, were tested to selectively target inflamed and injured cells in the brain. The targeting efficiency was visualized using a fluorescent imaging system (Thermo iBright FL1500) with liposomes labeled using a commercially available dye (calcein red; Sigma-Aldrich Co.). The captured images demonstrate successful delivery of the fluorescent dye into the inflamed mouse brain after restraint stress (FIGS. 2A-2F: brain images), revealing that the delivery can be achieved at the cellular level.

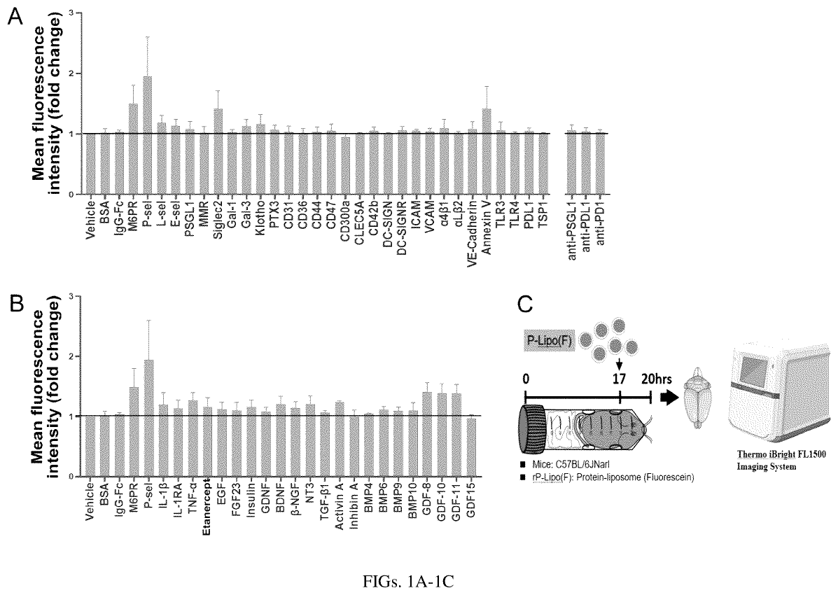

Corresponding quantitative analysis results are shown in FIG. 1 (FIGS. 1A, and 1B, brain fluorescence levels; 1C, experiment outline and settings). Groups with average fluorescence levels higher than those of BSA groups (black horizontal line) were considered more effective at delivery across the BBB, including P-sel, CI-MPR, L-selectin (L-sel), E-selectin (E-sel), P-selectin glycoprotein ligand-1 (PSGL-1), CD22 (siglec2), galectin-3 (Gal-3), Klotho, pentraxin 3 (PTX3), CD47, CD42b, dendritic cell-specific intercellular adhesion molecule-3-grabbing non-integrin (DC-SIGN) receptor (DC-SIGNR), intercellular adhesion molecule 1 (ICAM-1; CD54), vascular cell adhesion molecule (VCAM), integrin α4β1, VE-caderin, annexin V, toll-like receptor 3 (TLR3), programmed cell death 1 ligand 1 (PD-L1), anti-PSGL-1 immunoglobulin (anti-PSGL-1 Ig), anti-PDL1 Ig, IL-1β, IL-1RA, TNF-α, etanercept, EGF, fibroblast growth factor (FGF) 23, insulin, glial cell-line derived neurotrophic factor (GDNF), BDNF, β-NGF, NT-3, TGF-β1, activin A, BMP4, BMP6, BMP9, BMP-10, GDF-8, GDF-10, GDF-11. In contrast, other tested proteins, such as BSA, IgG-Fc, mannose receptor (MMR, CD206), galectin-1 (Gal-1), CD31, CD36, CD44, CD300a, CLEC5, DC-SIGN, integrin αLβ2, TLR4, TSP1, anti-PD1 Ig, inhibin A, GDF-15, did not show this effect in liposome cargo delivery across the BBB.

### Example 3: Engineered Liposomes Conjugated with Secretory Proteins Can Successfully Deliver Caspase-1 siRNA Into the Inflamed Mouse Brain Following Restraint Stress, Effectively Rescuing Pyroptosis in Mouse Brain Cells, Including Neurons, Glial Cells, and Astrocytes

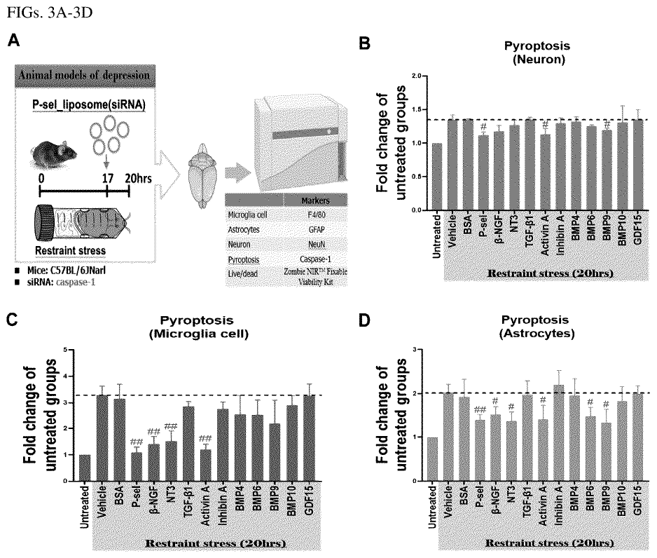

Engineered liposomes with secretory protein conjugations were designed to selectively target inflamed and injured cells in the brain. The targeting efficiency was visualized using a fluorescent imaging system (Thermo iBright FL1500) with liposomes labeled using a commercially available dye (calcein red; Sigma-Aldrich Co.). The captured images demonstrated successful delivery of the fluorescent dye into the inflamed mouse brain after restraint stress. FIG. 3A provides an overview of the experiment. The markers used to label neurons, microglia, astrocytes, and pyroptosis signal in the flow cytometry analyses were NeuN, F4/80, GFAP, and the activated form of caspase-1, respectively (FIG. 3A). FIGS. 3B, 3C, and 3D show the relative pyroptosis levels (a form of regulated cell death) in neurons, microglia, and astrocytes, respectively, in the mouse brain under restraint stress conditions compared to controls. #P<0.05 vs. BSA groups, indicating significant rescue. These findings align with the fluorescein delivery experiments (FIGS. 1 and 2), where P-selectin, b-NGF, NT3, TGF-b1, activin A, BMP4, BMP6, BMP9, and BMP10 demonstrate effective drug-delivery effect in neurons (B), microglia cells (C), and/or astrocytes (D) in the mouse brain following restraint stress. Additionally, siRNA is an ideal cargo for this drug-delivery system, as demonstrated in this experiment with caspase-1 siRNA, which effectively ameliorates pyroptosis and brain inflammation induced by restraint stress in mice.

### Example 4: Liposomes Bearing the Fluorescent Marker and Conjugated With Multiple Secretory Proteins (e.g., CI-MPR+KL, CI-MPR+P-Selectin, and KL+P-Selectin) Exhibited Markedly Heightened Fluorescence Compared to Cases Where Each Secretory Protein was Used Individually

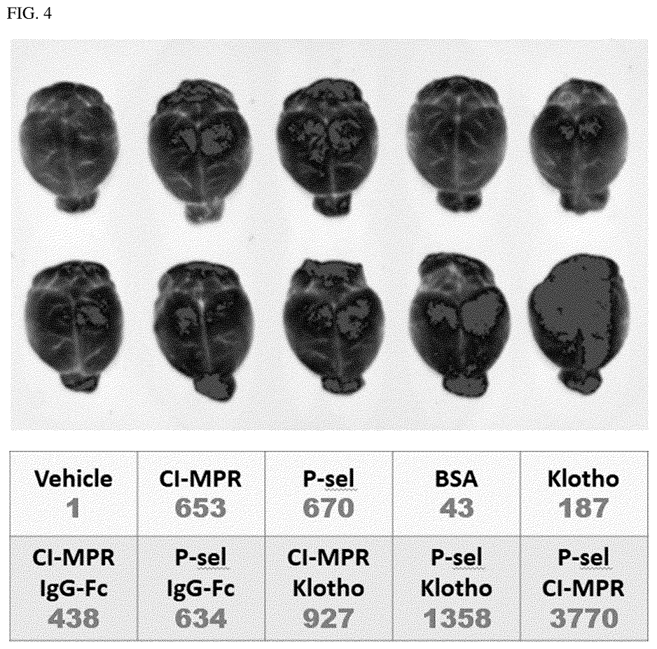

Engineered liposomes with secretory protein conjugations were designed to selectively target inflamed and injured cells in the brain. The targeting efficiency was visualized using a fluorescent imaging system (Thermo iBright FL1500) with liposomes labeled using a commercially available dye (calcein red; Sigma-Aldrich Co.). The captured images demonstrated successful delivery of the fluorescent dye into the inflamed mouse brain after restraint stress (FIG. 4). The secretory protein s utilized in this trial were itemized in FIG. 4, with the corresponding quantification of relative brain fluorescence levels provided in the same figure. This aligns with the observed synergistic effect demonstrated in the hepatitis-rescue experiments shown in FIG. 1. When liposomes bearing the fluorescent marker were conjugated with multiple secretory protein s (e.g., CI-MPR+KL, CI-MPR+P-selectin, and KL+P-selectin), they exhibited markedly heightened fluorescence compared to cases where each secretory protein was used individually. Once more, these findings point to the presence of a synergistic effect when employing a combination of these secretory protein s in tandem with the liposomes, even when employed for delivering a substance across the BBB.

### Example 5: Liposomes Conjugated with P-Selectin Were Able to Facilitate the Penetration of Caspase-3 Inhibitor (Z-DEVD-FMK)-Loaded Liposomes Across the BBB in Mice

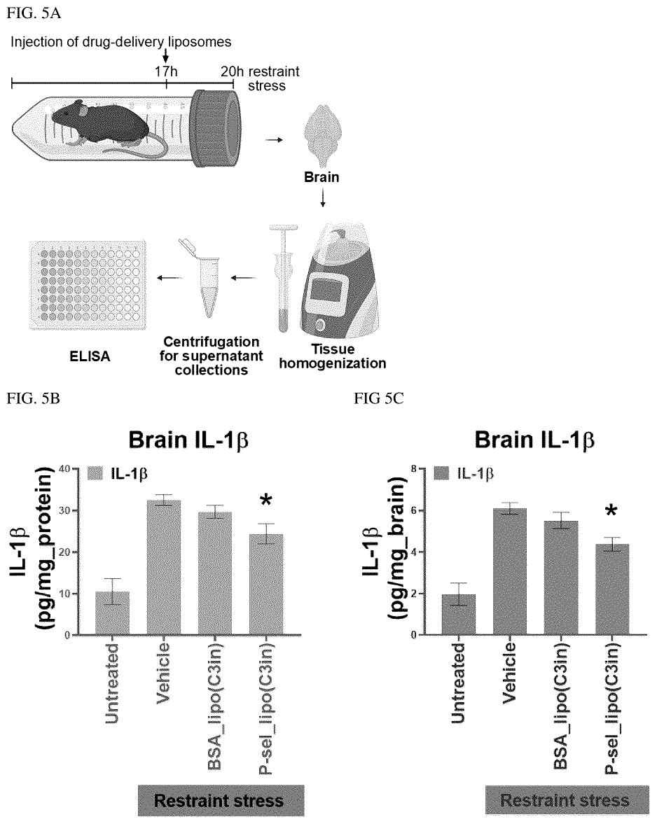

These secretory protein-conjugated liposomes were tested to specifically target inflamed and injured cells in the brain. Restraint stress induced inflammation in the mouse brain, as evidenced by the increased levels of the pro-inflammatory cytokine IL-1 (FIG. 5A: experiment outline; FIGS. 5B and 5C: IL-1 levels in untreated vs. restraint stress-vehicle groups). Notably, only liposomes conjugated with P-selectin and not control protein bovine serum albumin (BSA) were able to facilitate the penetration of caspase-3 inhibitor (z-DEVD-FMK)-loaded liposomes across the BBB in mice.

### Example 6: P-Selectin-Conjugated Liposomes Significantly Improved Lipopolysaccharide (LPS)-induced anxiety-and depression-like behavior in mice.

LPS-induced depression-like behavior. LPS-induced depression-like behavior was induced in mice using previously described methods (Hall, S.; Arora, D.; Anoopkumar-Dukie, S.; Grant, G. D. Effect of Coffee in Lipopolysaccharide-Induced Indoleamine 2,3-Dioxygenase Activation and Depressive-like Behavior in Mice. J Agric Food Chem 2016, 64, 8745-8754, doi:10.1021/acs.jafc.6b03568; O'Connor, J. C.; Lawson, M. A.; Andre, C.; Moreau, M.; Lestage, J.; Castanon, N.; Kelley, K. W.; Dantzer, R. Lipopolysaccharide-induced depressive-like behavior is mediated by indoleamine 2,3-dioxygenase activation in mice. Mol Psychiatry 2009, 14, 511-522, doi:10.1038/sj.mp.4002148).

Mouse behavioral test. A mouse behavioral test “open field test” was conducted to measure depression and anxiety-like behavior. These tests followed established protocols (Seibenhener, M. L. ; Wooten, M. C. Use of the Open Field Maze to measure locomotor and anxiety-like behavior in mice. J Vis Exp 2015, 10.3791/52434, e52434, doi:10.3791/52434; Lopatina, O.; Yoshihara, T.; Nishimura, T.; Zhong, J.; Akther, S.; Fakhrul, A. A. ; Liang, M.; Higashida, C.; Sumi, K.; Furuhara, K., et al. Anxiety- and depression-like behavior in mice lacking the CD157/BST1 gene, a risk factor for Parkinson's disease. Front Behav Neurosci 2014, 8, 133, doi:10.3389/fnbeh.2014.00133).

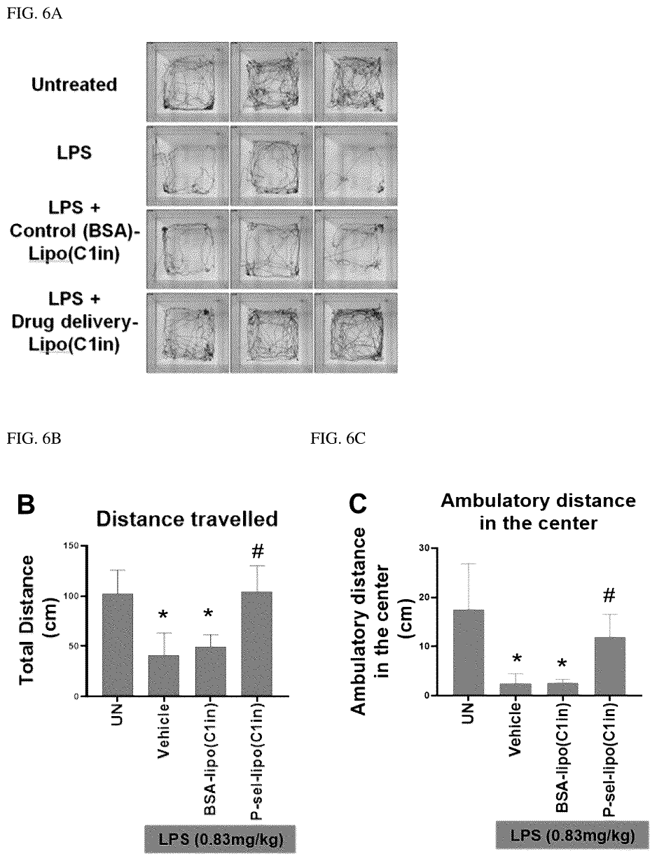

Based on the aforementioned findings, it has been established that restraint stress has the capability to induce brain inflammation. However, this brain inflammation can be mitigated by delivering anti-cell death agents using secretory protein-conjugated liposomes that can traverse the BBB. In light of these results, our objective is to further investigate whether the depressive behavior exhibited by mice can be alleviated through the treatment of secretory protein-conjugated liposomes loaded with a caspase-1 inhibitor. Our findings demonstrate that P-selectin-conjugated liposomes significantly improved lipopolysaccharide (LPS)-induced anxiety-and depression-like behavior in mice (FIG. 6A: records of mouse open field test; FIGS. 6B and 6C: quantitative analyses of mouse behavior). Notably, the intravenous administration of P-selectin-conjugated liposomes loaded with the caspase-1 inhibitor z-WEHD-FMK demonstrated a remarkable alleviation of LPS-induced depression-like behavior when compared to the vehicle groups (*P<0.05, P-sel-lipo (C1in) vs. vehicle groups). [P-sel-lipo(C1in): P-selectin-conjugated liposomes-loaded with caspase-1 inhibitor z-WEHD-FMK].

### Example 7: Caspase-1 Inhibitor-Loaded Liposomes Conjugated with P-Selectin Significantly Improved Restraint Stress-Induced Anxiety and Depression-Like Behavior

Mouse behavioral test. Mouse behavioral tests, including the open arm test (Horii, Y.; McTaggart, I.; Kawaguchi, M. Testing Animal Anxiety in Rats: Effects of Open Arm Ledges and Closed Arm Wall Transparency in Elevated Plus Maze Test. J Vis Exp 2018, 10.3791/56428, doi:10.3791/56428) and tail suspension test (Can, A.; Dao, D. T.; Terrillion, C. E.; Piantadosi, S. C.; Bhat, S.; Gould, T. D. The tail suspension test. J Vis Exp 2012, 10.3791/3769, e3769, doi:10.3791/3769), were conducted to measure depression and anxiety-like behavior in mice. These tests followed established protocols.

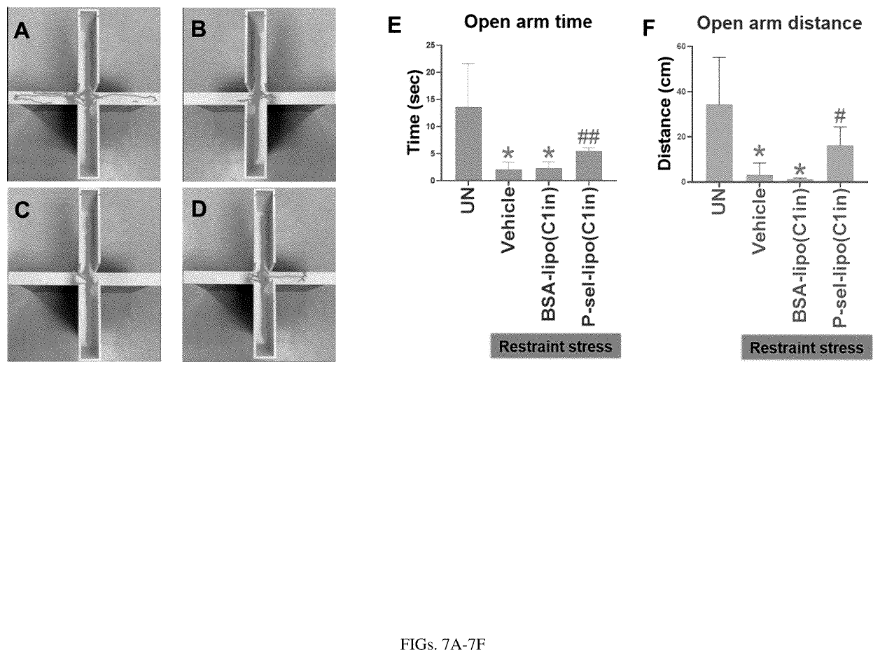

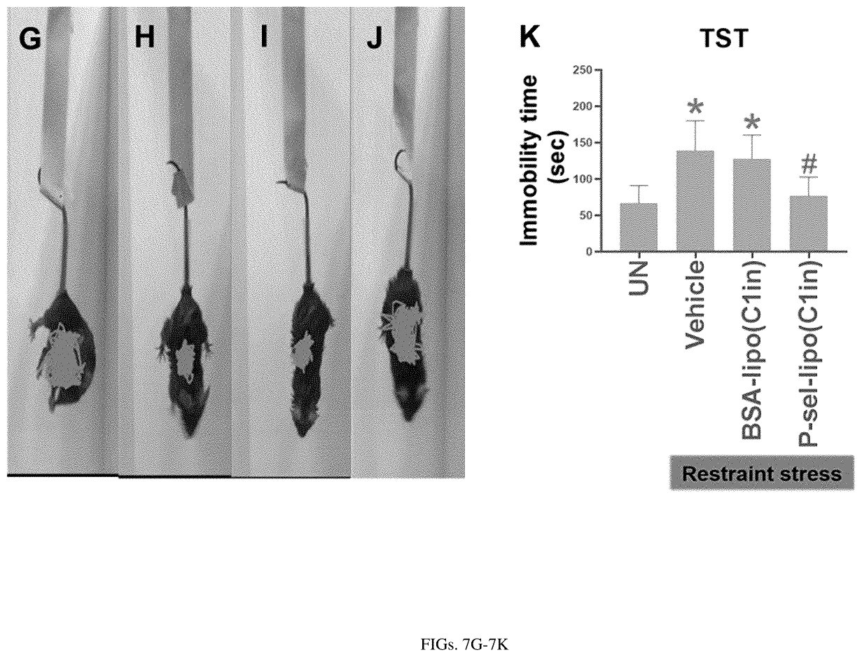

Our objective is to investigate whether secretory protein-liposomes loaded with drugs can mitigate anxiety-like and depression-like behaviors induced by restraint stress, following the established model for restraint stress-induced depression. Previous studies have shown that 20-hour restraint stress can lead to a depression-like phenotype. Based on this model, we conducted additional analyses using the Open Arm Test (OAT) and Tail Suspension Test (TST), which are well-recognized methods for assessing depression and anxiety-like behavior in mice. These tests were performed based on previously described methods. Our findings revealed that only caspase-1 inhibitor-loaded liposomes conjugated with P-selectin, and not BSA-conjugated liposomes, significantly improved restraint stress-induced anxiety and depression-like behavior (FIGS. 7A to 7F), consistent with the observations from the LPS model (FIG. 6). Once again, these results demonstrate the ability of this secretory protein-based drug delivery system to transport small molecules and peptide/short protein fragments into the brain and exert their pharmaceutical effects.

### Example 8: This Drug-Delivery System Can Successfully Deliver Large Functional Enzymes, Such as Cre DNA Recombinase, Across the Blood-Brain Barrier (BBB) Into the Mouse Brain. This Allows for Genome Editing and Alteration of Protein Expression, Demonstrated by the Shift from Red Fluorescent Protein Expression to Green Fluorescent Protein Expression

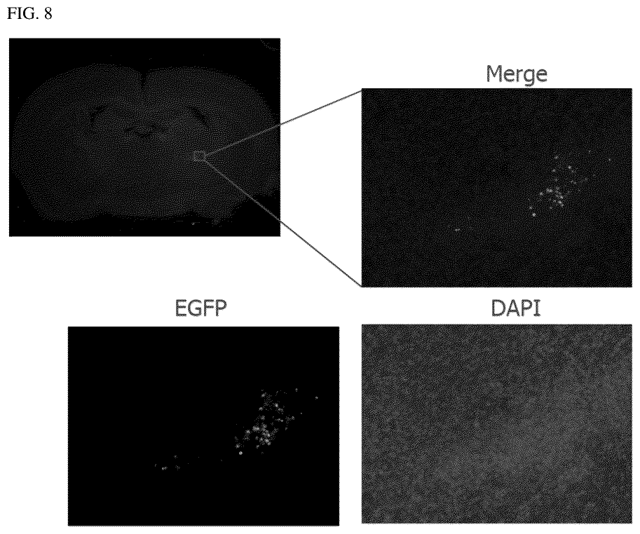

Here we employed a Cre-Lox recombination system to demonstrate the effective delivery of functional Cre-recombinase into the mouse brain. Specifically, we targeted a transgenic mouse [B6.129-(Gt(ROSA)26Sor tm4(ACTB-tdTomato,-EGFP)Luo/] using a vector designed with a CMV enhancer/chicken beta-actin core promoter (pCA) to drive the expression of a loxP-flanked, N-terminal membrane-tagged, optimized DsRed fluorescent protein variant known as tandem-dimer-Tomato (tdTomato), followed by a polyadenylation signal. Adjacent to the second loxP site, there was an N-terminal membrane-tagged enhanced green fluorescent protein (EGFP) sequence, also followed by a polyadenylation signal. Additionally, an frt-flanked neo cassette was positioned distally to the expression vector. Red fluorescence was observed in all tested tissues. When exogenous Cre-recombinase was delivered to mouse brain cells, the floxed region was excised, enabling the expression of the EGFP cassette. Consequently, these cells that received Cre-recombinase became labeled with EGFP (FIG. 8). These results indicate that this delivery system is well-suited for the efficient delivery of functional enzymes and proteins, such as those used in enzyme replacement therapy, to brain cells without apparent lysosome digestion.

### Example 9: Caspase-1 siRNA-Loaded Liposomes Conjugated with P-Selectin Significantly Improved Restraint Stress-Induced Anxiety and Depression-Like Behavior

Mouse behavioral test. Mouse behavioral test of open field maze (OFM) (Michael L. Seibenhener and Michael C. Wooten Use of the Open Field Maze to Measure Locomotor and Anxiety-like Behavior in Mice. J Vis Exp. 2015; (96): 52434; doi: 10.3791/52434 PMCID: PMC4354627 PMID: 25742564) was conducted to measure depression and anxiety-like behavior in mice. This test followed established protocols.

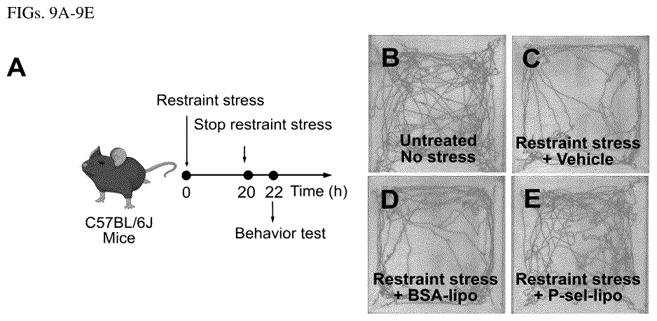

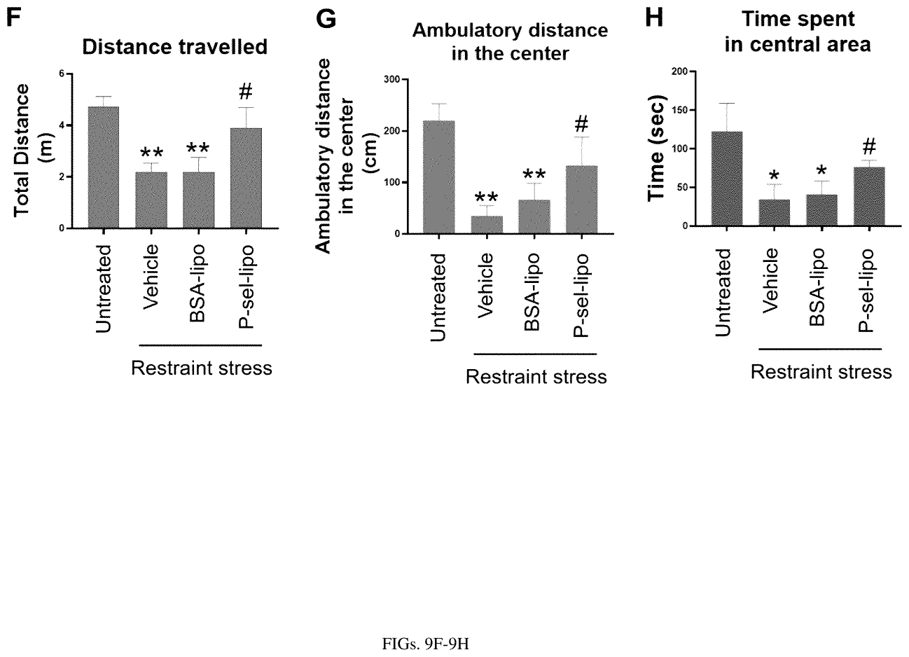

We aim to further investigate whether secretory protein-liposomes loaded with drugs can alleviate anxiety-like and depression-like behavior induced by restraint stress, following the restraint stress-induced depression model. It has been reported that 24-hour restraint stress can induce a depression-like phenotype. In accordance with this model, we conducted additional analyses using the OFM test, which is established methods for measuring depression and anxiety-like behavior in mice. This test was performed based on previously described methods. Our findings revealed that only caspase-1 siRNA-loaded liposomes conjugated with P-selectin, and not BSA-conjugated liposomes, significantly improved restraint stress-induced anxiety and depression-like behavior (FIGS. 9A, experiment outline, 9B to 9H). Once again, these results demonstrate the ability of this secretory protein-based drug delivery system to transport small molecules and short nucleotide fragments into the brain and exert their pharmaceutical effects.

1. A method for delivery of an agent across a biological barrier to a cell and/or a tissue containing the cell, comprising contacting a protein-vesicle conjugate or a pharmaceutical comprising the protein-vesicle conjugate with a cell and/or a tissue of a subject, wherein the protein-vesicle conjugate comprises:
(a) one or more secretory proteins or a fragment thereof expressed on or conjugated to a surface of a vesicle, and/or an antibodies specific to the one or more secretory proteins or the fragment thereof and expressed on or conjugated to the surface of the vesicle; and
(b) an agent embedded in the vesicle.

2. The method of claim 1, wherein the cell is an inflamed and/or injured cell.

3. The method of claim 1, wherein the biological barrier is selected from intestinal barrier, nasal barrier, pulmonary barrier, blood-brain barrier (BBB), and skin barrier.

4. The method of claim 1, wherein the biological barrier is the blood-brain barrier (BBB).

5. The method of claim 1, wherein before administration of the protein-vesicle conjugate, the method further comprises a step of inducing and/or administering the inflamed and/or injured cell within the BBB.

6. The method of claim 1, wherein the one or more secretory proteins or the fragment thereof is selected from P-selectin (P-sel), cation-independent mannose 6-phosphate receptor (CI-MPR), L-selectin (L-sel), E-selectin (E-sel), P-selectin glycoprotein ligand-1 (PSGL-1), CD22 (siglec2), galectin-3 (Gal-3), Klotho, pentraxin 3 (PTX3), CD47, CD42b, dendritic cell-specific intercellular adhesion molecule-3-grabbing non-integrin (DC-SIGN) receptor (DC-SIGNR), intercellular adhesion molecule 1 (ICAM-1; CD54), vascular cell adhesion molecule (VCAM), integrin α4β1, VE-caderin, annexin V, toll-like receptor 3 (TLR3), programmed cell death 1 ligand 1 (PD-L1), IL-1β, IL-1RA, TNF-α, etanercept, EGF, fibroblast growth factor (FGF) 23, insulin, glial cell-line derived neurotrophic factor (GDNF), BDNF, β-NGF, NT-3, TGF-β1, activin A, BMP4, BMP6, BMP9, BMP-10, GDF-8, GDF-10, GDF-11, and a fragment thereof.

7. The method of claim 1, wherein the one or more antibodies specific to the one or more secretory proteins of the fragment thereof is selected from anti-PSGL-1 immunoglobulin (anti-PSGL-1 Ig) and anti-PDL1 Ig.

8. The method of claim 1, wherein the cell is a neuron, and the one or more secretory proteins or fragment thereof are selected from P-selectin, activin A, BMP9, and a fragment thereof.

9. The method of claim 1, wherein the cell is a microglia cell, and the one or more secretory proteins or the fragment thereof are selected from P-selectin, β-NGF, NT3, activin A, and a fragment thereof.

10. The method of claim 1, wherein the cell is an astrocyte, and the one or more secretory proteins or the fragment thereof are selected from P-selectin, β-NGF, NT3, activin A, BMP6, BMP9, and a fragment thereof.

11. The method of claim 8, wherein the protein-vesicle conjugate comprises CI-MPR in combination with Klotho, CI-MPR in combination with P-selectin, or Klotho in combination with P-selectin.

12. The method of claim 1, wherein the vesicle is a liposome or a micelle.

13. The method of claim 1, wherein the protein-vesicle conjugate is artificially engineered or cell-derived.

14. The method of claim 1, wherein the protein-vesicle conjugate is cell-derived.

15. The method of claim 1, wherein the agent is separated from the secretory proteins or the fragment thereof and encapsulated within the vesicle or attached to an outer surface of the vesicle.

16. The method of claim 1, wherein the agent is a diagnostic contrast agent, a cell survival enhancing agent, a cell survival suppressing agent, a cell component, an organelle, a cell, a cytotoxic agent, an antitumor drug, a toxin, an antibody, a lipid, a protein, DNA, RNA, a therapeutic agent or a nanomaterial.

17. The method of claim 1, which is directed to a disease associated with regulated cell death of brain cell alteration.

18. The method of claim 17, wherein the disease associated with regulated cell death of brain cell alteration is trauma, exposure to chemical and physical toxic factors, genetic disease, age-related disease, cardiovascular disorder associated with brain damage, infectious disease, neoplastic disease, neurodegenerative disease, metabolic disease, aging, obesity, cancer, inflammation, psychological stress, stroke, neurodegeneration induced by β-amyloid or α-synuclein or toxicity, myodegenerative conditions, or chronic lung inflammation caused by cystic fibrosis, or autoimmune disease.

19. The method of claim 17, wherein the neurodegenerative disease is brain fog, Alzheimer's disease, Parkinson's disease, Huntington's disease, Amyotrophic Lateral Sclerosis or stroke, the cardiovascular disorder associated with brain damage is ischemia, heart failure or infectious disease, and the autoimmune disease is systemic lupus erythematosus, autoimmune lymphoproliferative syndrome, rheumatoid arthritis or thyroiditis.
